(19) United States
(12) Patent Application Publication
(10) Pub. No.: US 2008/0118992 A1
(43) Pub. Date: May 22, 2008

(54) Method For Measuring Molecular Interactions By Laser Light Scattering (Lls)

(75) Inventors: Tommaso Bellini, Milano (IT); Andrea Ghetta, Milano (IT); Marco Buscaglia, Milano (IT)

Correspondence Address: YOUNG & THOMPSON, 209 Madison Street, Suite 500, ALEXANDRIA, VA, 22314, US

(73) Assignee: UNIVERSITA DEGLI STUDI DI MILANO, MILANO ITALY (IT)

(21) Appl. No.: 11/663,224

(22) PCT Filed: Sep. 16, 2005
(86) PCT No.: PCT/EP05/09955

§ 371 Date: Apr. 3, 2007

(30) Foreign Application Priority Data
Sep. 21, 2004 (IT) M12004A001800

(51) Int. Cl. G01N 33/543

(52) U.S. Cl. 436/518

(57) ABSTRACT

Procedure for the quantitative determination of interactions of ligands with receptors adsorbed on the surface of particles, by direct measure of the scattering of light by Laser Light Scattering (LLS), with the usage of submicrometric polymeric particles with diameter between 5 and 200 nm.

The present invention relates to a simple and effective method for the quantitative determination of ligand interactions with receptors adsorbed on the particle surface by means of direct light scattering measurement.

More specifically, the present invention relates to a method for the quantitative determination of interactions of ligand with receptors wherein submicrometric polymeric particles, having a diameter between 5 and 200 nm, preferably having particle sizes between 40 and 80 nm, are used.

Several methods have been suggested in the prior art to determine interactions between ligands and receptors, i.e. the binding affinities of ligand-receptor reversible systems of chemical, biochemical or biological interest. A list of the main methods is reported in Angew. Chem. Int. Ed. 1998, 37, page 2785.

Said known methods generally comprise the receptor immobilization on a suitable flat surface and the determination of variations of the properties, for example the optical ones, of said surface after having put it into contact with the ligands, said variations being induced by the formation of receptor-ligand couples.

One class of methods requires the ligand labeling in solution, i.e. the covalent ligand modification with fluorescent, luminescent or radioactive species. See for example the patent application US 2004/0014060 A1.

However the ligand modification is a very complex and long operation and it can hardly be used in screening tests wherein a notable variety of ligands is used. Furthermore the method requires an additional removal operation from the system, by washing out the free ligands, i.e. those which have not interacted with the receptors and which interfere with the measurement.

A further drawback of said method is that the ligand-receptor interaction can be influenced by the chemical modification of the ligand due to the labeling.

Another class of methods which more effectively simulate the receptor-ligand interactions, for example those occurring on a cell membrane surface, is the one which directly utilizes the variations induced on a surface by the bond formation in the receptor-ligand couple without modifying the ligand with labeling substances. An example of said method is the one which uses the BIAcore biosensor, commercialized by Pharmacia Biosensor AB (Uppsala, Sweden) described for example in the patents U.S. Pat. No. 5,313,264 and U.S. Pat. No. 5,374,563.

In this biosensor, based on the principle of Surface Plasmon Resonance (SPR) (J. Homola et al. “Surface Plasmon Resonance Sensors: review”, Sensor and Actuators B 54 (1999) 3-15), an evanescent optical wave couples with surface plasmons having thin layers (50 nm) of conductor materials such as silver or gold and generates a resonance phenomenon at specific angles. This allows to determine the variation of the refractive index of the layer adsorbed on the metal, for example a ligand-receptor couple. From this variation the binding constants between ligand and receptor are obtained.

Said method, even if often used in practice, is rather complex and expensive and is not always accurate in the determination of the binding constants. See for example the publication “Use of surface plasmon resonance to probe the equilibrium and dynamic aspects of interactions between biological macromolecules”, by Peter Schuck, Annu. Rev. Biophys. Biomol. Struct., 1997, 26; pages 541-66. The problems related to the use of the BIAcore method for the binding constant determination depend on:
1) the ligand mass transport which influences the determination;
2) the steric hindrance of the ligand-receptor couple (bulk effect) and the distribution of the binding sites on the sensor, which influence the adsorption and desorption constants. Therefore, not rarely, the association and dissociation constants obtained with this method differ of some orders of magnitude from those drawn by other methods;
3) the fact that the measurements are not taken under thermodynamic equilibrium conditions (kinetic approach).

The need was therefore felt to have available a method for the determination of interactions between ligands and receptors directly exploiting the variations induced by the ligand-receptor interaction on a surface, avoiding the ligand labeling and washing operations, and being able to act under thermodynamic equilibrium conditions, avoiding the drawbacks of the kinetic methods such as for example BIAcore.

It has now been surprisingly and unexpectedly found that it is possible to obviate the above mentioned drawbacks with a quantitative optical method which allows to determine the binding affinities of molecular species in thermodynamic equilibrium by means of the method described hereinafter.

It is object of the present invention a method for the determination of the binding constant of two interacting molecular species by means of Laser Light Scattering (LLS), comprising the following steps:
a) addition to a colloidal aqueous suspension or latex containing from 0.05% to 5% by weight of particles having an average diameter comprised between 5 and 200 nm, constituted by an hydrophobic amorphous polymer having a refractive index nₚ comprised between 1.3250 and 1.3400, preferably between 1.3300 and 1.3350—of a sequence of known volumes of an aqueous solution of a mixture containing from 50% to 99.5% by weight of an amphiphilic non ionic surfactant, or in case ionic as well, and from 0.5 to 50% by weight of the same or of a different surfactant ended with a receptor, measuring after each addition the intensity of the light scattered by the suspension by Laser Light Scattering (LLS) and reporting it on a diagram in connection with the progressively added solution volume, until reaching an asymptotic value Iᵣ;
b) addition to the suspension obtained in step a) of a sequence of known volumes of an aqueous solution of ligands, expressing as [T₀] the molar concentration of ligands added to the suspension, measuring after each addition the intensity of the light I scattered by the suspension by Laser Light Scattering (LLS) and reporting it on a diagram in connection with the progressively added solution volume, until reaching an asymptotic value and fitting the scattered light intensity data in connection with the ligand additions using equation 1, said equation being:

$$I = {{I}_{0} ( \sqrt{\frac{{I}_{r}}{{I}_{0}}} + \frac{\begin{matrix}{m}_{l} ( {n}_{l}^{2} - {n}_{w}^{2} ) ( [ {T}_{0} ] + {K}^{- 1} + [ {S}_{0} ] - \\ \sqrt{{( [ {T}_{0} ] + {K}^{- 1} + [ {S}_{0} ] )}^{2} - 4 [ {T}_{0} ] [ {S}_{0} ]} )\end{matrix}}{2 {ρ}_{l} ( {n}_{p}^{2} - {n}_{w}^{2} ) {φ}_{p}} )}^{2} \qquad ( 1 )$$

where I₀ is the intensity of light scattered by uncovered particles, n_{w} is the solvent refractive index, n₁ is the refractive index of ligands, φₚ is the fraction of suspension volume occupied by the particles, ρ₁ is the density of pure ligand, m₁ is the molecular weight of ligand molecule, [S₀] is the total molar concentration of ligand-receptor interaction sites and K is the binding constant.

Equation 1, used to fit the data of scattered light intensity in connection with the ligand additions, is derived by the Rayleigh model for the intensity of light scattered by particles much smaller than the wavelength (see for example “Light Scattering by Small Particles” H. C. van de Hulst, Dover Publications, Inc. New York) and by a function known as “Langmuir isotherm” which states the ligand amount bound to the receptor in connection with the added ligand amount [T₀], the receptor concentration [S₀] and the affinity constant K (see for example “Principles of Colloid and Surface Chemistry”, P. C. Hiemenz, Marcel Dekker Inc.). Since the other magnitudes involved are known, from fitting it is possible to draw the concentration of receptor adsorbed on the particles surface [S₀] and the affinity constant K for the ligand-receptor interaction.

The amorphous hydrophobic polymer can be, for example, a perfluoropolymer.

As amphiphilic surfactants those generating a self assembled monolayer on the latex particles are used. The obtainment of said monolayer can be achieved by carrying out the step a) of the present method by using only the non ionic, or in case ionic as well, surfactant in place of its mixture with the surfactant ended with the receptor and observing the reaching of an asymptotic value of the diagram.

Furthermore said non functionalized surfactants must not have specific interactions, i.e. they must not form a bond with the ligand to be analyzed. The absence of such inter-action can be verified by carrying out the first step of the method according to the invention by using only the surfactant and not the mixture, and following step b), verifying that there are no variations of the scattered light intensity.

According to the invention, non ionic surfactants (either glycolipids or surfactants of the family of oxyethylenes (brij)) can be used either as molecules carrying the sites acting as receptors or as “spacers” non interacting on the particle surface; otherwise ionic surfactants can be used, too: for example anionic, like bis(2-ethylhexyl) sulfosuccinate sodium salt (AOT, produced by Sigma), or cationic, like didecyldimethylammonium bromide (DDAB, produced by Sigma as well).

Among non ionic surfactants usable in the present invention it can be mentioned for example:
a) non ionic compounds having structure

CH₃—(CH₂)ₙ—(OCH₂CH₂)ₘOH

wherein 6<n<18 and 3<m<12

for example the commercial compound Brij 56 (Fluxa, cas. No. 9004-95-9) wherein n=15 and m is distributed around the value m=10;
b) alkyl glycosides with the following structure

RO—(CH₂)ₙ—CH₃

wherein 6<n<12 and R=glucose or maltose residue, for example the commercial compound n-dodecyl-beta-D-maltoside by Aldrich.

The amphiphilic surfactants ended with a receptor are prepared by reaction of the above described surfactants with receptors according to known prior art processes.

The receptor-ligand couple is defined as a molecule couple, for example proteins, nucleic acids, glycoproteins, carbohydrates, hormones, having an affinity suitable to produce a more or less stable bond. In particular antibody/antigen, enzyme/inhibitor, carbohydrate/carbohydrate, protein/DNA, DNA/DNA, peptide/peptide, can be mentioned.

In steps a) and b) of the method according to the invention the measurements of the scattered light intensities are carried out under thermodynamic equilibrium conditions, i.e. alternating the additions with periods of time, generally 4-6 minutes, in order to allow the suspension to stabilize.

It has been found that the invention system quickly reaches the thermodynamic equilibrium. Therefore the measurements carried out are independent from the absorption-desorption kinetics and thus are not influenced anyway by the bulk transport.

The configuration of the colloidal system with submicrometric particles makes available a larger surface in comparison with the systems which utilize flat surfaces, a solution volume being fixed. Generally, the diameter of the polymer particles and the concentration of the colloidal aqueous suspension of polymer are chosen in order to have an available surface per milliliter of suspension comprised between 500 and 2000 cm².

The method of the present invention allows to detect up to 3 micrograms of material per milliliter, corresponding to a sensitivity limit on the adsorbed mass per surface of 0.04 nanograms/mm² which is in the range of the most sensitive techniques of the prior art.

It is surprising and unexpected that the scattering of light (LS) has resulted effective to identify and measure interactions between receptors and ligands according to the method of the present invention. In fact, the interaction of ligands with receptors in diluted solutions is not measurable by LS.

On the contrary the use of submicrometric particles which support a multiplicity of receptors allows to use LS to determine the ligand-receptor interaction.

It is necessary to note that the presence of interactions between a ligand and more receptors carried by different particles (indicated herein as polyvalent interactions) makes the present invention method inapplicable. In the case of several polyvalent interactions one can reach the latex coagulation. The existence of said polyvalent interactions can be verified by determining the particle size during steps a) and b) by the Dynamic Laser Light Scattering (DLLS) technique. The DLLS method is based on the registration of a curve which binds the scattering intensity and the release time of the scattering articles. It is thus possible to draw a release rate Γ, which is proportional to the scattering coefficient D of the scattering species:

Γ=D*q²

wherein q represents the wave vector which is expressed as follows:

q=(4πn/λ)sin(θ/2)

wherein n is the medium refraction index, λ is the wave length and θ is the scattering angle at which the measurements are carried out.

The scattering coefficient D is related to the diameter of the present scattering articles by the Stokes-Einstein equation:

D=kT/3πηφ

wherein K is the Boltzmann constant, T the temperature, η the medium viscosity and φ the diameter of the scattering articles. Therefore from this equation the particle diameter can be drawn. In absence of polyvalent interactions the polymeric particle diameter remains substantially constant. The diameter variation is due to the monomolecular layer formed by the surfactant, by the receptor and by the ligand.

The control of diameter variation is particularly important when there is no system coagulation, even if polyvalent interactions are present. In this case, indeed, the obtained measurements would not be significant of the ligand-receptor interactions.

Therefore the interactions which must take place between receptor and ligand must not be polyvalent interactions. The diameter variation for interactions which are not polyvalent is of the order of few nanometers for supporting polymer particles of about 40 nm. There are, instead, polyvalent interactions when, for example, particles of 80 nm are detected using supporting particles of 40 nm.

Some Examples are given for illustrative but not limitative purposes of the present invention.

### EXAMPLES

### Example 1

### Determination of the Binding Constant between Vancomycin Hydrochloride Hydrate (Ligand) and the Peptide Sequence L-Lys-D-Ala-D-Ala (Receptor)

Step a)

To a colloidal aqueous suspension containing 0.1% by weight of submicrometric particles having an average diameter of 78 nm, constituted by a TFE copolymer containing 40% by moles of perfluoromethylvinylether, it was added a 10 millimolar aqueous solution of a mixture containing 99% by weight of n-dodecyl-beta-D-maltoside and 1% by weight of the non ionic surfactant Brij 56 ended with the peptide sequence L-Lys-D-Ala-D-Ala, sequence characteristic of the bacterium cellular wall, each in 6 microliter portions, at intervals of 5 min.

After each addition the mixture was stirred for 30 seconds and let balance for 1 minute, and the scattering light intensity was measured by using a 5 milliwatt He—Ne laser and a photomultiplier to convert the scattered light into an electric signal.

The light intensity was recorded for 10 seconds for consecutive six times thus selecting the lowest value in order to minimize the noise due to the possible presence of powder in the sample.

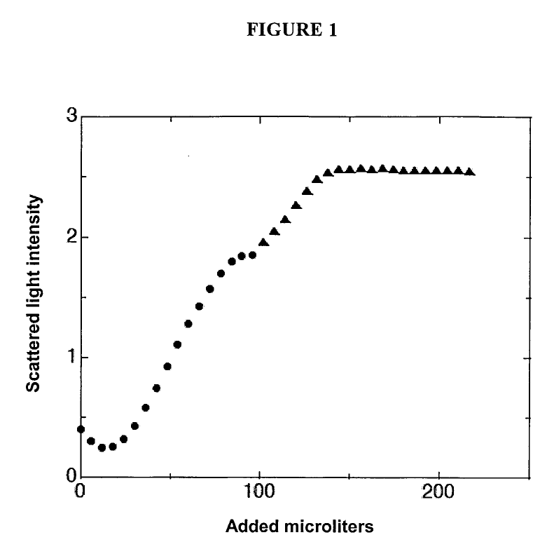

The measured intensity values (spots in FIG. 1) are represented as a diagram in connection with the added solution volumes obtaining the curve reported in FIG. 1.

The progressive particle covering by the used mixture is monitored by the variation of the scattered light intensity.

The complete coating is clearly shown by the achievement of an asymptotic value of the scattered light intensity.

Step b)

To the suspension obtained in a), when the asymptotic value is reached, a 0.4 millimole aqueous solution of Vancomycin hydrochloride hydrate (commercialized by Aldrich, cas. No. 861987) is added, each in 6 microliter portions, at intervals of 5 minutes.

After each addition the mixture was stirred for 30 seconds and let balance for 1 minute, and the scattered light intensity was measured as in step a).

The measured intensity values (triangles in FIG. 1) are represented as a diagram in connection with the solution volumes and added to the curve diagrammed in step a).

The formation of the Vancomycin/L-Lys-D-Ala-D-Ala couples is detected from the increase of the scattered light intensity until reaching an asymptotic value which indicates the saturation of the receptor sites with Vancomycin.

By fitting the Langmuir absorption formula to the scattered light intensity data, in connection with the Vancomycin additions, the receptor-ligand binding constant is obtained.

The obtained binding constant is 1.5×10⁶ moles⁻¹.

In order to verify the absence of aggregation processes, the diameter of the submicrometric particles was continuously checked by means of the DLLS method and, substantially, it remained constant.

### Example 2

The Example 1 was repeated but using an aqueous colloidal suspension at 0.1% of particles having an average diameter of 40 nm, constituted by a TFE copolymer containing 30% by moles of 2,2,4-trifluoro-5-trifluoromethoxy-1,3-dioxole (TTD).

In step a) the same mixture of the Example 1 was added in 12 microliter portions.

In step b) a 0.9 millimolar mixture of Vancomycin was added in 6 microliter portions.

The obtained binding constant is 1.1×10⁶ moles⁻¹.

1. Method for the determination of the binding constant of two interacting molecular species by means of Laser Light Scattering (LLS), comprising the following steps:
a) addition to a colloidal aqueous suspension or latex containing from 0.05% to 5% by weight of particles having an average diameter comprised between 5 and 200 nm, constituted by an hydrophobic amorphous polymer having a refractive index nₚ comprised between 1.3250 and 1.3400, preferably between 1.3300 and 1.3350—of a sequence of known volumes of an aqueous solution of a mixture containing from 50% to 99.5% by weight of an amphiphilic non ionic surfactant, or in case ionic as well, and from 0.5 to 50% by weight of the same or of a different surfactant ended with a receptor, measuring after each addition the intensity of the light scattered by the suspension by Laser Light Scattering (LLS) and reporting it on a diagram in connection with the progressively added solution volume, until reaching an asymptotic value Iᵣ;
b) addition to the suspension obtained in step a) of a sequence of known volumes of an aqueous solution of ligands, expressing as [T₀] the molar concentration of ligands added to the suspension, measuring after each addition the intensity I of the light scattered by the suspension by Laser Light Scattering (LLS) and reporting it on a diagram in connection with the progressively added solution volume, until reaching an asymptotic value and fitting the scattered light intensity data in connection with the ligand additions using equation 1, said equation being:
$$I = {{I}_{0} ( \sqrt{\frac{{I}_{r}}{{I}_{0}}} + \frac{\begin{matrix}{m}_{l} ( {n}_{l}^{2} - {n}_{w}^{2} ) ( [ {T}_{0} ] + {K}^{- 1} + [ {S}_{0} ] - \\ \sqrt{{( [ {T}_{0} ] + {K}^{- 1} + [ {S}_{0} ] )}^{2} - 4 [ {T}_{0} ] [ {S}_{0} ]} )\end{matrix}}{2 {ρ}_{l} ( {n}_{p}^{2} - {n}_{w}^{2} ) {φ}_{p}} )}^{2} \qquad ( 1 )$$
where I₀ is the intensity of light scattered by uncovered particles, n_{w} is the solvent refractive index, n₁ is the refractive index of ligands, φₚ is the fraction of suspension volume occupied by the particles, □₁ is the density of pure ligand, m₁ is the molecular weight of ligand molecule, [S₀] is the total molar concentration of ligand-receptor interaction sites and K is the binding constant.

2. Method according to claim 1 wherein the non ionic, or ionic, amphiphilic surfactants are surfactants which produce a monolayer (self assembled monolayer) on the polymer particles.

3. Method according to claim 1 wherein the non ionic surfactants are chosen among:
a) non ionic compounds having the structure
CH₃—(CH₂)ₙ—(OCH₂CH₂)ₘOH
where 6<n<18 and 3<m<12
b) alchil glycosides with the following structure
RO—(CH₂)ₙ—CH₃
where 6<n<12 and R=glucose or maltose residue.

4. Method according to claim 1 wherein the ligand-receptor couple is chosen among proteins, nucleic acids, glycoproteins, carbohydrates, hormones.

5. Method according to claim 4 wherein the ligand-receptor couple of molecules is chosen among antibody/antigen, enzyme/inhibitor, carbohydrate/carbohydrate, protein/DNA, DNA/DNA, peptide/peptide.

6. Method according to claim 1 wherein the diameter of the polymer particles and the concentration of the colloidal aqueous suspension of polymer are chosen in order to have an available surface per milliliter of suspension comprised between 500 and 2000 cm².

7. Method according to claim 2 wherein the non ionic surfactants are chosen among:
a) non ionic compounds having the structure
CH₃—(CH₂)ₙ—(OCH₂CH₂)ₘOH
where 6<n<18 and 3<m<12
b) alchil glycosides with the following structure
RO—(CH₂)ₙ—CH₃
where 6<n<12 and R=glucose or maltose residue.

8. Method according to claim 2 wherein the ligand-receptor couple is chosen among proteins, nucleic acids, glycoproteins, carbohydrates, hormones.

9. Method according to claim 3 wherein the ligand-receptor couple is chosen among proteins, nucleic acids, glycoproteins, carbohydrates, hormones.

10. Method according to claim 2 wherein the ligand-receptor couple is chosen among proteins, nucleic acids, glycoproteins, carbohydrates, hormones.

11. Method according to claim 3 wherein the ligand-receptor couple is chosen among proteins, nucleic acids, glycoproteins, carbohydrates, hormones.

12. Method according to claim 4 wherein the ligand-receptor couple is chosen among proteins, nucleic acids, glycoproteins, carbohydrates, hormones.

13. Method according to claim 5 wherein the ligand-receptor couple is chosen among proteins, nucleic acids, glycoproteins, carbohydrates, hormones.
